(19) United States
(12) Patent Application Publication
(10) Pub. No.: US 2026/0234519 A1
(43) Pub. Date: Aug. 13, 2026

(54) PHOTOSYNTHETIC MICROORGANISM CULTURE METHOD AND ASSOCIATED DEVICE

(71) Applicant: CARBONWORKS, LIBOURNE (FR)

(72) Inventor: Philippe BOUDIER, Cadaujac (FR)

(73) Assignee: CARBONWORKS, LIBOURNE (FR)

(21) Appl. No.: 19/149,152

(22) PCT Filed: Jan. 25, 2024
(86) PCT No.: PCT/FR2024/050107

§ 371 Date: Jul. 18, 2025

(30) Foreign Application Priority Data
Jan. 25, 2023 (FR) FR2300675

(51) Int. Cl. C12M 1/00; C12M 1/40; C12N 1/12; C12N 9/88; C12N 11/14; C12R 1/89

(52) U.S. Cl. CPC C12M 21/02; C12M 21/18; C12M 43/06; C12N 1/12; C12N 9/88; C12N 11/14; C12R 2001/89; C12Y 402/01001

(57) ABSTRACT

A method of culturing photosynthetic microorganisms in a biological reactor including a culture medium. The carbon source of the culture media has at least one inorganic carbon, and the inorganic carbon is entirely in the form of bicarbonate ions (HCO₃—) or in the form of HCO₃— ions and carbon dioxide. The method also includes a step of enzymatically converting CO₂ to HCO₃— with enzymes immobilised on a support.

### FIELD

The present invention relates to a method for culturing photosynthetic microorganisms in a biological reactor comprising a culture medium in which the carbon source comprises at least one inorganic carbon, said inorganic carbon being entirely in the form of bicarbonate ions (HCO3-) or in the form of HCO3- ions and carbon dioxide. In particular, the method according to the invention comprises a step of enzymatically converting CO2 to HCO3- by means of enzymes immobilised on a support.

### BACKGROUND

Culturing microorganisms can be made on a laboratory scale in Petri or Erlen dishes, but also on a larger, industrial, scale in biological reactors of 50,000 litres and more.

Many photosynthetic microorganisms, especially microalgae, are thus cultured for intrinsic properties of the biomasses obtained. For example, culturing algae or some microalgae makes it possible to produce biomasses rich in lipids and/or proteins. These biomass are then used for the preparation of nutrient, cosmetic and pharmaceutical compositions, or for the preparation of biofuel or biofertiliser.

These photosynthetic microorganisms use inorganic carbon, particularly in the form of carbon dioxide (CO2) and bicarbonate ions (HCO3-) to grow and multiply.

However, the ability to develop reactors allowing industrial culture of photosynthetic microorganisms with effective absorption of inorganic carbon brought into the culture medium, in particular the ability to bring a sufficient amount of inorganic carbon assimilable by photosynthetic microorganisms without affecting culture conditions (especially pH variations), remains to be improved.

The purpose of the present invention is to meet this need.

### SUMMARY

The present invention relates to a method for culturing photosynthetic microorganisms with bicarbonate ions (HCO3-) as the main carbon source in a culture system comprising a biological reactor, a carbon dioxide (CO2) supply device and a module for enzymatically converting CO2 to HCO3- ions coupled to the reactor, said method comprising the steps of:
enriching the culture medium with CO2 via the CO2 supply device, to obtain a CO2 enriched culture medium, and
contacting the CO2 enriched culture medium obtained with CO2 to HCO3- conversion enzymes in the conversion module to obtain a HCO3- ion enriched culture medium, in which conversion module the CO2 to HCO3- conversion enzymes are immobilised on a support.

The invention also relates to a system for culturing photosynthetic microorganisms comprising a biological reactor, a device for supplying carbon dioxide (CO2) and a module for enzymatically converting CO2 to bicarbonate ions (HCO3-) coupled to the reactor, said conversion module comprising enzymes for converting CO2 to HCO3- ions immobilised on a support.

### BRIEF DESCRIPTION OF THE DRAWINGS

FIG. 1: Culture mode of the microorganisms with a reactor (1), a CO2 supply device (2) disposed between the reactor and the enzymatic conversion module (3).

FIG. 2: Membrane contactor (B) and cross-section view of a membrane of the membrane contactor (A).

FIG. 3A: Enzymatic conversion module according to one embodiment of the invention in which the enzymes are immobilised on a support in the form of plates coated with enzymes.

FIG. 3B: A more specific embodiment of the invention, in which the enzyme-coated plates are integrated into in a tube.

FIG. 4: Embodiment of the invention in which the enzymes are immobilised on a support placed in an independent enclosure (53), the enclosure being disposed between the CO2 supply device (52) and the biological reactor (51).

### DETAILED DESCRIPTION

The present invention relates to a method for culturing photosynthetic microorganisms with HCO3- ions as the main carbon source in a culture system comprising a biological reactor, a carbon dioxide (CO2) supply device and a module for enzymatically converting CO2 to HCO3-ions coupled to the reactor, said method comprising the steps of:
enriching the culture medium with CO2 via the CO2 supply device, to obtain a CO2 enriched culture medium, and
contacting the CO2 enriched culture medium obtained with CO2 to HCO3-conversion enzymes in the conversion module to obtain a HCO3- ion enriched culture medium, in which conversion module the CO2 to HCO3- conversion enzymes are immobilised on a support.

### Definitions

By “carbon source”, it is meant a nutrient brought into the culture medium and providing carbon necessary for the formation of new organic molecules and the chemical reactions of molecularly synthesising the microorganism cultured. This carbon source can be inorganic (CO2, HCO3-) or organic (sugars, fatty acids).

By “main carbon source”, it is meant a carbon source representing more than 50%, in particular more than 60%, more than 70%, more than 80%, more than 90%, or even about 100% of the carbon source in the culture medium.

By “HCO3- ions as the main carbon source”, it is meant that in the culture medium, more than 50%, in particular, more than 60%, more than 70%, more than 80%, more than 90%, or even about 100% of the carbon source is in the form of HCO3- ions.

By “biological reactor”, it is meant a reactor within which biological phenomena develop, such as the growth of a single type of microorganism or of a consortium of microorganisms (in particular microalgae), in very varied fields such as effluent treatment, production of biomass containing biomolecules of interest (i.e. biomolecules able to be valued) and/or metabolite products.

For the purposes of the present invention, a device is said to be “coupled” to the biological reactor when it constitutes a separate element of the reactor but is connected to this reactor by means of a fluidic network in which the culture medium included in the reactor can circulate.

By “culture medium”, it is meant a liquid medium comprising the nutrients essential for the development, growth and/or multiplication of the cultured microorganisms. Nutrients essential for the development of photosynthetic microorganisms are known per se, and can be adapted in amount and nature especially depending on the microorganism(s) cultured, the size of the reactor, the amount of biomass desired. These nutrients include, for example, sources of carbon, nitrogen, oxygen, phosphorus, etc.; minerals such as calcium, iron, magnesium, potassium, sodium, sulphur, etc.; trace elements (boron, zinc, copper, cobalt, etc.)

By “CO2 enriched culture medium”, it is meant a culture medium with a dissolved CO2 content of between 200 and 600 mg/L.

By “HCO3- enriched culture medium”, it is meant a culture medium whose dissolved HCO3- content is between 200 and 600 mg/L.

By “Photosynthetic microorganisms”, it is meant eukaryotic or prokaryotic microorganisms that use light as an energy source and inorganic carbon, in the form of CO2 or HCO3-, as the main carbon source. These include bacteria, protists such as yeasts or microalgae.

By “protist”, it is meant all eukaryotic unicellular microorganisms including microalgae, yeasts and protozoa. By “autotrophic culture conditions”, it is meant a culture medium comprising both water, an inorganic carbon source and light. By “inorganic carbon”, it is meant carbon species including carbon dioxide, carbonic acid as well as bicarbonate and carbonate anions.

By “hydrogel”, it is meant a matrix comprising a network of polymers that swells in contact with water and which comprises between 75 and 90% water by weight.

### Microorganisms

The microorganisms cultured according to the invention are photosynthetic microorganisms, eukaryotes such as protists or procaryotes such as bacteria.

In one embodiment, the cultured microorganisms are bacteria especially cyanobacteria and in particular cyanobacteria of the Anabaena, Nostoc, Microcistis, Arthrospira and Spirulina genera.

According to one preferred embodiment, the cultured microorganisms are protists, even more preferably microalgae.

In one embodiment, the microorganisms according to the invention are microalgae preferably belonging to the families of Chlorophytes, Rodophytes, Bigyra and Haptophytes.

When the microorganisms belong to the family of Chlorophytes, they are selected from microorganisms belonging to the class of Chlorophyceae, Ulvophyceae, Trebouxiophyceae, Prasinophyceae, Pedinophyceae and Charophyceae.

In one embodiment, the microorganisms belong to the class of Chlorophyceae, and more particularly to the order of Sphaeropleales. The microorganisms are then preferably of the Scenedesmus genus.

When the microalgae are of the Scenedesmus genus, they may be selected from species S. abundans, S. aciculatus, S. aculeolatus, S. aculeotatus, S. acuminatus, S. acutiformis, S. acutus, S. aldavei, S. alternans, S. ambuehlii, S. anhuiensis, S. anomalus, S. antennatus, S. antillarum, S. apicaudatus, S. apiculatus, S. arcuatus, S. aristatus, S. armatus, S. arthrodesmiformis, S. arvernensis, S. asymmetricus, S. bacillaris, S. baculiformis, S. bajacalifornicus, S. balatonicus, S. basiliensis, S. bernardii, S. bicaudatus, S. bicellularis, S. bidentatus, S. bijuga, S. bijugatus, S. bijugus, S. brasiliensis, S. breviaculeatus, S. brevispina, S. caribeanus, S. carinatus, S. caudato-aculeolatus, S. caudatus, S. chlorelloides, S. circumfusus, S. coalitus, S. costatogranulatus, S. crassidentatus, S. curvatus, S. decorus, S. denticulatus, S. deserticola, S. diagonalis, S. dileticus, S. dimorphus, S. disciformis, S. dispar; S. distentus, S. ecornis, S. ellipsoideus, S. ellipticus, S. falcatus, S. fenestratus, S. flavescens, S. flexuosus, S. furcosus, S. fuscus, S. fusiformis, S. gracilis, S. graevenitzii, S. grahneisii, S. granulatus, S. gujaratensis, S. gutwinskii, S. hanleyi, S. helveticus, S. heteracanthus, S. hindakii, S. hirsutus, S. hortobagyi, S. houlensis, S. huangshanensis, S. hystrix, S. incrassatulus, S. indianensis, S. indicus, S. inermis, S. insignis, S. intermedius, S. javanensis, S. jovais, S. jugalis, S. kerguelensis, S. kissii, S. komarekii, S. lefevrei, S. linearis, S. littoralis, S. longispina, S. longus, S. luna, S. lunatus, S. magnus, S. maximus, S. microspina, S. minutus, S. mirus, S. morzinensis, S. multicauda, S. multiformis, S. multispina, S. multistriatus, S. naegelii, S. nanus, S. notatus, S. nygaardii, S. oahuensis, S. obliquus, S. obtusiusculus, S. obtusus, S. olvalternus, S. oocystiformis, S. opoliensis, S. ornatus, S. ovalternus, S. pannonicus, S. papillosum, S. parisiensis, S. parvus, S. pecsensis, S. pectinatus, S. perforatus, S. planctonicus, S. plarydiscus, S. platydiscus, S. pleiomorphus, S. polessicus, S. polydenticulatus, S. polyglobulus, S. polyspinosus, S. praetervisus, S. prismaticus, S. producto-capitatus, S. protuberans, S. pseudoarmatus, S. pseudobernardii, S. pseudodenticulatus, S. pseudogranulatus, S. pseudohystrix, S. pyrus, S. quadrialatus, S. quadricauda, S. quadricaudata, S. quadricaudus, S. quadrispina, S. raciborskii, S. ralfsii, S. reginae, S. regularis, S. reniformis, S. rostrato-spinosus, S. rotundus, S. rubescens, S. scenedesmoides, S. schnepfii, S. schroeteri, S. securiformis, S. semicristatus, S. semipulcher; S. sempervirens, S. senilis, S. serrato-perforatus, S. serratus, S. serrulatus, S. setiferus, S. sihensis, S. smithii, S. soli, S. sooi, S. spicatus, S. spinoso-aculeolatus, S. spinosus, S. spinulatus, S. striatus., S. subspicatus, S. tenuispina, S. terrestris, S. tetradesmiformis, S. transilvanicus, S. tricostatus, S. tropicus, S. tschudyi, S. vacuolatus, S. variabilis, S. velitaris, S. verrucosus, S. vesiculosus, S. westii, S. weberi, S. wisconsinensis, S. wuhanensis, S. wuhuensis. Advantageously according to the invention, algae of the Scenedesmus genus may be algae selected from species S. obliquus or S. bundans.

In another embodiment, the microorganisms belong to the class of Chlorophyceae, and more particularly to the order of Chlamydomonadales. The microorganisms are then preferably of the Haematococcus genus.

When microalgae are of the Haematococcus genus, they may be selected from the following species: H. buetschlii, H. capensis, H. carocellus, H. droebakensis, H. grevilei, H. insignis, H. lacustris, H. murorum, H. pluvialis, H. salinus, H. sanguineis, H. thermalis, H. Zimbabwe.

In another embodiment, the microorganisms belong to the class of Trebouxiophyceae, and more particularly to the order of Chlorellales. The microorganisms are preferably of the Chlorella genus.

When the microalgae are of the Chlorella genus, they may be selected from species C. acuminata, angustoellipsoidea, anitrata, antarctica, C. aureoviridis, C. autotrophica, C. botryoides, C. caldaria, C. candida, C. capsulata, C. chlorelloides, C. cladoniae, C. coelastroides, C. colonialis, C. communis, C. conductrix, C. conglomerata, C. desiccata, C. ellipsoidea, C. elongata, C. emersonii, C. faginea, C. fusca, C. glucotropha, C. homosphaera, C. infusionum, C. kessleri, C. koettlitzii, C. lacustris, C. lewinii, C. lichina, C. lobophora, C. luteo-viridis, C. marina, C. miniata, C. minor; C. minutissima, C. mirabilis, C. mucosa, C. mutabilis, C. nocturna, C. nordstedtii, C. oblonga, C. oocystoides, C. ovalis, C. paramecii, C. parasitica, C. parva, C. peruviana, C. photophila, C. pituita, C. pringsheimii, C. protothecoides, C. pulchelloides, C. pyrenoidosa, C. regularis, C. reisiglii, C. reniformis, C. rotunda, C. rubescens, C. rugosa, C. saccharophila, C. salina, C. simplex, C. singularis, C. sorokiniana, C. spaerckii, C. sphaerica, C. stigmatophora, C. subsphaerica, C. terricola, C. trebouxioids, C. vannielii, C. variabilis, C. viscosa, C. volutis, C. vulgaris, C. zopfingiensis. Advantageously, according to the invention, the Chlorella genus may be selected from species C. sorokiniana or C. vulgaris.

In another embodiment, the microorganisms belong to the family of Chlorophytes, but to the class of Chlorodendrophyceae. In this embodiment, the microorganisms are of the Tetraselmis genus, and are selected from species: T. alacris, T. apiculata, T. arnoldii, T. ascus, T astigmatica, T. bichlora, T. bilobata, T. bolosiana, T. chui, T. contracta, T. convolutae, T. cordiformis, T. desikacharyi, T. elliptica, T. fontiana, T. gracilis, T. hazenii, T. helgolandica, T. impellucida, T. incisa, Inconspicuous T, T. indica, T. levis, T. maculata, Marine, Mediterranean, Micropapilla, Rubens, T. striata, T. subcordiformis, T. sueca, T. tetrabrachia, T. tetrathele, T. verrucosa, T. viridis, T. wettsteinii.

When the microorganisms belong to the family of Rhodophytes, they are preferably selected from the microorganisms belonging to the class of Cyaniophyceae. Mention may be made in particular of microalgae belonging to the order of Cyanidiales and more particularly to the family of Cyanidiaceae and the family of Galdieriaceae. Examples of microorganisms are microorganisms of the Cyanidioschyzon genus such as Cyandidioschyzon merolae, Cyanidium such as Cyanidium caldarium, Cyanidium daedalum, Cyanidium maximum, Cyanidium partitum and Cyanidium rumpens, Galdieria such as Galdieria daedala, Galdieria maxima, Galdieria partita and Galdieria sulphuraria, Pluto including Pluto calderium.

The microorganisms may additionally be selected from microorganisms belonging to the super-class of Dinoflagellates. Among these microorganisms, mention may be made in particular of microorganisms of the Amphidinium genus, such as species Amphidinium acutum, Amphidinium aloxalocium, Amphidinium asymmetricum, Amphidinium bipes, Amphidinium carbunculus, Amphidinium carterae, Amphidinium carteri, Amphidinium celestinum, Amphidinium coeruleum, Amphidinium conradi, Amphidinium corallinum, Amphidinium crassum, Amphidinium cyaneoturba, Amphidinium dubium, Amphidinium elegans, Amphidinium extensum, Amphidinium flagellans, Amphidiniumflexum, Amphidiniumfusiforme, Amphidinium glaucum, Amphidinium globosum, Amphidinium hoefleri, Amphidinium hyalinum, Amphidinium incoloratum, Amphidinium inflatum Amphidinium kesslitzi, Amphidinium klebsii, Amphidinium lacustre, Amphidinium lanceolatum, Amphidinium latum, Amphidinium lilloense, Amphidinium longum, Amphidinium luteum, Amphidinium machapungarum, Amphidinium macrocephalum, Amphidinium mammillatum, Amphidinium massartii, Amphidinium microcephalum, Amphidinium operculatum, Amphidinium ornithocephalum, Amphidinium ovoideum, Amphidinium ovum, Amphidinium pellucidum, Amphidinium phthartum Skuja, Amphidinium psammophilum, Amphidinium pseudogalbanum, Amphidinium purpureum, Amphidinium salinum Ruinen, Amphidinium schroederi, Amphidinium scissoides, Amphidinium sphenoides, Amphidinium steini, Amphidinium stellatum, Amphidinium subsalsum, Amphidinium tortum, Amphidinium trochodinoides, Amphidinium turbo, Amphidinium vasculum, Amphidinium vittatum and Amphidinium wislouchii.

In another embodiment, the microorganisms are selected from the class of Euglenoidea, and more particularly from microalgae of the Euglena genus. They may be selected, among others, from species E. viridis, E. gracilis, E. limosa, E. globosa, E. prowsei, E. polomorpha.

When the microalgae are diatoms, they may be selected in particular from the Nitzschia, Navicula, Gyrosigma, Phaeodactylum, Thalassiosira genera.

When the microalgae are of the Nitzschia genus, they may be selected in particular from species N. abbreviata, N. abonuensis, N. abridia, N. accedens, N. accomodata, N. aciculariformis, N. acicularioides, N. acicularis (including all these varieties), N. acidoclinata, N. actinastroides, N. actydrophila, N. acula, N. acuminata (including all these varieties), N. acuta, N. adamata, N. adamatoides, N. adapta, N. adducta, N. adductoides, N. admissa, N. admissoides, N. aequalis, N. aequatorialis, N. aequora, N. aequorea, N. aerophila, N. aerophiloides, N. aestuari, N. affinis, N. africana, N. agnewii, Agnite, Alba, Albicostalis, Alexandrin, N. alicae, N. allanssonii, N. alpina, N. alpinobacillum, N. amabilis, N. ambigua, N. americana, N. amisaensis, N. amphibia, N. amphibia (including all these varieties),
N. amphibioides, N. amphicephala, N. amphilepta, N. amphioxoides, N. amphioxys (including all these varieties), N. amphiplectans, N. amphiprora, N. amplectens, N. amundonii, N. anassae, N. andicola, N. angularis (including all these varieties), N. angulata, N. angustata (including all these varieties), N. angustatula, N. angustiforaminata, N. aniae, N. antarctica, N. antillarum, N. apiceconica, No. beehives, No. archibaldii, No. arcuata, No. arcula, N. arcus, N. ardua, N. aremonica, N. arenosa, N. areolata, N. armoricana, N. asperula, N. astridiae, N. atomus, N. attenuata, N. aurantiaca, N. aurariae, N. aurica, N. auricula, N. australis, N. austriaca, N. bacata (including all these varieties), N. bacillariaeformis, N. bacilliformis, N. bacillum, N. balatonis, N. balcanica, N. baltica, N. barbieri (including all these varieties),
N. barkleyi, N. barronii, N. barrowiana, N. bartholomei, N. bathurstensis, N. bavarica, N. behrei, N. bergii, N. beyeri, N. biacrula, N. bicapitata (including all these varieties), bicuneata, N. bifurcata, N. bilobata (including all these varieties), N. birostrata, N. bisculpta, N. bita, N. bizertensis, N. blankaartensis, N. bombiformis, N. borealis, N. bosumtwiensis, N. braarudii, N. brebissonii (including all these varieties), N. bremensis (including all these varieties), N. brevior; N. brevirostris, N. brevissima (including all these varieties), N. brevistriata, N. brightwellii, N. brittonii, N. brunoi, N. bryophila, N. buceros, N. bukensis, N. bulnheimiana, N. buschbeckii, N. calcicola, N. caledonensis, N. calida (including all these varieties), N. californica, N. campechiana, N. capensis, N. capitata, N. capitellata (including all these varieties), N. capuluspalae, N. carnicobarica, N. carnico-barica, N. challengeri, N. chalonii, N. chandolensis, N. chardezii, N. chasei, N. chauhanii, N. chungara, N. chutteri, N. circumsuta, N. clarissima, N. clausii, N. clementei, N. clementia, N. clevei, N. closterium (including all these varieties), N. coarctata, N. cocconeiformis, N. communis (including all these varieties),
N. commutata, N. commutatoides, N. compacta, N. compressa (including all these varieties), N. concordia, N. confinis, N. conformata, N. confusa, N. congolensis, N. constricta (including all these varieties), N. consummata, N. corpulenta, N. costei, N. coutei, N. creticola, N. cucumis, N. cursoria, N. curta, N. curvata, N. curvilineata, N. curvipunctata, N. curvirostris (including all these varieties), N. curvula (including all these varieties), N. cuspidata, N. cylindriformis, N. cylindrus, N. dakariensis, N. davidsonii, N. dealpina, N. debilis, N. decipiens, N. delauneyi, N. delicatissima, N. delicatula, N. delognei, N. denticula (including all these varieties), N. denticuloides, N. desertorum, N. dianae, N. diaphana, N. diducta, N. didyma, N. dietrichii, N. dilatata, N. diluviana, N. dippelii, N. directa, N. diserta, N. disputata, N. dissipata (including all these varieties), N. dissipatoides, N. distans (including all these varieties),
N. distantoides, N. divaricata, N. divergens, N. diversa, N. diversecostata, N. doljensis, N. draveillensis, N. droebakensis, N. dubia (including all these varieties), N. dubiformis, N. dubioides, N. ebroicensis, N. eglei, N. elegans, N. elegantula, N. elegens, N. elliptica, N. elongata, N. entomon, N. epiphytica, N. epiphyticoides, N. epithemiformis, N. epithemioides, N. epithemoides (including all these varieties), N. epsilon, N. erlandssonii, N. erosa, N. etoshensis, N. examinanda, N. eximia, N. famelica, N. fasciculata, N. febigeri, N. ferox, N. ferrazae, N. fibula-fissa, N. filiformis (including all these varieties), N. flexa, N. flexoides, N. fluminensis, N. fluoresces, N. fluvialis, N. fogedii, N. fonticola (including all these varieties), N. fonticoloides, N. fonticula, N. fontifuga, N. forfica, N. formosa, N. fossalis, N. fossilis, N. fragilariiformis, N. franconica, N. fraudulenta, N. frauenfeldii, N. frequens, N. frickei, N. frigida (including all these varieties),
N. frustuloides, N. frustulum (including all these varieties), N. fruticosa, N. fundi, N. fusiformis, N. gaarderi, N. gaertnerae, N. gandersheimiensis, N. garrensis, N. gazellae, N. geitleri, N. geitlerii, N. gelida (including all these varieties), N. geniculata, N. gessneri, N. gieskesii, N. gigantea, N. gisela, N. glabra, N. glacialis (including all these varieties), N. glandiformis, N. goetzeana (including all these varieties), N. gotlandica, N. graciliformis, N. gracilis (including all these varieties), N. gracillima, N. graciloides, N. gradifera, N. graeffii, N. grana, N. grandis, N. granii (including all these varieties), N. granulata (including all these varieties), N. granulosa, N. groenlandica, N. grossestriata, N. grovei, N. gruendleri, N. grunowii, N. guadalupensis, N. guineensis, guttula, gyrosigma, habirshawii, habishawii, N. hadriatica, N. halteriformis, N. hamburgiensis, N. hantzschiana (including all these varieties), N. harderi, N. harrissonii, N. hassiaca, N. heidenii, N. heimii, N. hemistriata, N. heteropolica, N. heuflerania, N. heufleriana (including all these varieties),
N. hiemalis, N. hiengheneana, N. hierosolymitana, N. hoehnkii, Holastica, hollerupensis, holsatica, homburgiensis, N. hudsonii, N. hummii, N. hungarica (including all these varieties), N. hustedti, N. hustedtiana, N. hyalina, N. hybrida (including all these varieties), N. hybridaeformis, N. ignorata (including all these varieties), N. iltisii, N. impressa, N. improvisa, N. incerta, N. incognita, N. inconspicua, N. incrustans, N. incurva (including all these varieties), N. indica, N. indistincta, N. inducta, N. inflatula, N. ingenua, N. inimasta, N. innominata, N. insecta, N. insignis (including all these varieties), N. intermedia (including all these varieties), N. intermissa, N. interrupta, N. interruptestriata, N. invicta (including all these varieties), N. invisa, N. invisitata, N. iranica, N. irregularis, N. irremissa, N. irrepta, N. irresoluta, N. irritans, N. italica, N. janischii, N. jelineckii, N. johnmartinii, N. juba, N. jucunda, N. jugata (including all these varieties),
N. jugiformis, N. kahlii, N. kanakarum, N. kanayae, N. kavirondoensis, N. kerguelensis, N. kimberliensis, N. kittlii, N. kittonii, N. knysnensis, N. kolaczeckii, N. kotschyi, N. kowiensis, N. krachiensis, N. krenicola, N. kuetzingiana (including all these varieties), N. kuetzingii, N. kuetzingioides, N. kurzeana, N. kurzii, N. kutzingiana (including all these varieties), N. labella, N. labuensis, N. lacrima, N. lacunarum, N. lacunicola, N. lacus-karluki, N. lacustris, N. lacuum, N. laevis, N. laevissima, N. lagunae, N. lagunensis, N. lamprocampa (including all these varieties), N. lanceola (including all these varieties), N. lanceolata (including all these varieties), N. lancettula, N. lancettuloides, N. lange-bertalotii, N. latens, N. latestriata, N. latiuscula, N. lauenbergiana, N. lauenburgiana, N. lecointei, N. leehyi, N. legleri, N. lehyi, N. leistikowii, N. lesbia, N. lesinensis, N. lesothensis, N. leucosigma, N. levidensis (including all these varieties),
N. liebetruthii (including all these varieties), N. ligowskii, N. limicola, N. limulus, N. linearis (including all these varieties), N. lineata, N. lineola, N. linkei, N. lionella, N. littoralis (including all these varieties), N. littorea, N. longa, N. longicollum, N. longirostris, N. longissima (including all these varieties), N. lorenziana (including all these varieties), N. lucisensibilis, N. lunaris, N. lunata, N. lurida, N. luzonensis, N. macaronesica, N. macedonia, N. macera, N. machardyae, N. macilenta (including all these varieties), N. magnacarina, N. mahihaensis, N. mahoodii, N. maillardii, N. major; N. majuscula (including all these varieties), N. makarovae, N. manca, N. mancoides, N. manguini, N. marginata, N. marginulata (including all these varieties), N. marina, N. martiana, N. maxima, N. media, N. medioconstricta, N. mediocris, N. mediterranea, N. metzeltinii, N. microcephala (including all these varieties),
N. migrans, N. minuta, N. minutissima, N. minutula, N. miramarensis, N. miserabilis, N. mitchelliana, N. modesta, N. moissacensis (including all these varieties), N. mollis, N. monachorum, N. monoensis, N. montanestris, N. morosa, N. multistriata, N. nana, N. natalensis, N. natans, N. nathorsti, N. navicularis, N. navis-varingica, N. navrongensis, N. neglecta, N. nelsonii, N. neocaledonica, N. neoconstricta, N. neofrigida, N. neogena, N. neotropica, N. nereidis, N. nicobarica, N. nienhuisii, N. normannii, N. notabilis, N. nova, N. novae-guineaensis, N. novae-guineensis, N. novaehollandiae, N. nova-zealandia, N. nyassensis, N. oberheimiana, N. obesa, N. obliquecostata, N. obscura, N. obscurepunctata, N. obsidialis, N. obsoleta, N. obsoletiformis, N. obtusa (including all these varieties), N. obtusangula, N. oceanica, N. ocellata, N. oliffi, N. omega, N. osmophila, N. ossiformis, N. ostenfeldii, N. ovalis, N. paaschei, N. pacifica, N. palacea, N. palea (including all these varieties), N. paleacea, N. paleaeformis, N. paleoides, N. palustris, N. pamirensis, N. panduriformis (including all these varieties), N. pantocsekii, N. paradoxa (including all these varieties), N. parallela, N. pararostrata, N. partita, N. parvula (including all these varieties), N. parvuloides, N. paxillifer; N. peisonis, N. pelagica, N. pellucida, N. pennata, N. peragallii, N. perindistincta, N. perminuta, N. perpusilla (including all these varieties), N. perspicua, N. persuadens, N. pertica, N. perversa, N. petitiana, N. philippinarum, N. pilum, N. pinguescens, N. piscinarum, N. plana (including all these varieties),
N. planctonica, N. plicatula, N. plioveterana, N. polaris, N. polymorpha, N. ponciensis, N. praecurta, N. praefossilis, N. praereinholdii, N. princeps, N. procera, N. prolongata (including all these varieties), N. prolongatoides, N. promare, N. propinqua, N. pseudepiphytica, N. pseudoamphioxoides, N. pseudoamphioxys, N. pseudoamphyoxys, N. pseudoatomus, N. pseudobacata, N. pseudocapitata, N. pseudocarinata, N. pseudocommunis, N. pseudocylindrica, N. pseudodelicatissima, N. pseudofonticola, N. pseudohungarica, N. pseudohybrida, N. pseudonana, N. pseudoseriata, N. pseudosigma, N. pseudosinuata, N. pseudostagnorum, N. pubens, N. pulcherrima, N. pumila, N. punctata (including all these varieties), N. pungens (including all these varieties), N. pungiformis, N. pura, N. puriformis, N. pusilla (including all these varieties), N. putrida, N. quadrangula, N. quickiana, N. rabenhorstii, N. radicula (including all these varieties), N. rautenbachiae, N. recta (including all these varieties),
N. rectiformis, N. rectilonga, N. rectirobusta, N. rectissima, N. regula, N. reimeri, N. reimerii, N. reimersenii, N. retusa, N. reversa, N. rhombica, N. rhombiformis, N. rhopalodioides, N. richterae, N. rigida (including all these varieties), N. ritscheri, N. robusta, N. rochensis, N. rolandii, N. romana, N. romanoides, N. romanowiana, N. rorida, N. rosenstockii, N. rostellata, N. rostrata, N. ruda, N. rugosa, N. rupestris, N. rusingae, N. ruttneri, N. salinarum, N. salinicola, N. salpaespinosae, N. salvadoriana, N. sansimoni, N. sarcophagum, N. scabra, N. scalaris, N. scaligera, N. scalpelliformis, N. schoenfeldii, N. schwabei, N. schweikertii, N. scutellum, N. sellingii, N. semicostata, N. semirobusta, N. separanda, N. seriata (including all these varieties), N. serpenticola, N. serpentiraphe, N. serrata, N. sibula (including all these varieties), N. sigma (including all these varieties), N. sigmaformis, N. sigmatella, N. sigmoidea (including all these varieties), N. silica, N. silicula (including all these varieties),
N. siliqua, N. similis, N. simplex, N. simpliciformis, N. sinensis, N. sinuata (including all these varieties), N. smithii, N. sociabilis, N. socialis (including all these varieties), N. solgensis, N. solida, N. solita, N. soratensis, N. sp., N. spathulata (including all these varieties), N. speciosa, N. spectabilis (including all these varieties), N. sphaerophora, N. spiculoides, N. spiculum, N. spinarum, N. spinifera, N. stagnorum, N. steenbergensis, N. stellata, N. steynii, N. stimulus, N. stoliczkiana, N. stompsii (including all these varieties), N. strelnikovae, N. stricta, N. strigillata, N. striolata, N. subaccommodata, N. subacicularis, N. subacuta, N. subamphioxioides, N. subapiculata, N. subbacata, N. subcapitata, N. subcapitellata, N. subcohaerens (including all these varieties), N. subcommunis, N. subconstricta, N. subcurvata, N. subdenticula, N. subfalcata, N. subfraudulenta, N. subfrequens, N. subfrustulum, N. subgraciloides, N. subinflata, N. subinvicta, N. sublaevis, N. sublanceolata, N. sublica, N. sublinearis, N. sublongirostris, N. submarina, N. submediocris, N. subodiosa, N. subpacifica, N. subpunctata, N. subromana, N. subrostrata, N. subrostratoides, N. subrostroides, N. subsalsa, N. subtilioides, N. subtilis (including all these varieties),
N. subtubicola, N. subvitrea, N. suchlandtii, N. sulcata, N. sundaensis, N. supralitorea, N. tabellaria, N. taenia, N. taeniiformis, N. tantata, N. tarda, N. taylorii, N. temperei, N. tenella, N. tenerifa, N. tenuiarcuata, N. tenuirostris, N. tenuis (including all these varieties), N. tenuissima, N. tergestina, N. terrestris, N. terricola, N. thermalis (including all these varieties), N. thermaloides, N. tibetana, N. tirstrupensis, N. tonoensis, N. towutensis, N. translucida, N. tropica, N. tryblionella (including all these varieties), N. tsarenkoi, N. tubicola, N. tumida, N. turgidula, N. turgiduloides, N. umaoiensis, N. umbilicata, N. umbonata, N. vacillata, N. vacua, N. valdecostata, N. valdestriata, N. valens, N. valga, N. valida (including all these varieties), N. vanheurckii, N. vanoyei, N. vasta, N. ventricosa, N. vermicularioides, N. vermicularis (including all these varieties), N. vermicularoides, N. vexans, N. victoriae, N. vidovichii, N. vildaryana, N. villarealii, N. virgata, N. visurgis, N. vitrea (including all these varieties), N. vivax (including all these varieties), N. vixnegligenda, N. vonhauseniae, N. vulga, N. weaveri, N. weissflogii, N. westii, N. williamsiii, N. wipplingeri, N. witkowskii, N. wodensis, N. woltereckii, N. woltereckoides, N. wuellerstorfii, N. wunsamiae, N. yunchengensis, N. zebuana, N. zululandica.

According to one particular embodiment of the invention, the protists are selected from the Chlorella, Galdieria and Euglena genera or cyanobacteria and diatoms.

### Biological Reactor

Biological reactors generally consist of a vessel possibly equipped with a stirring or agitating element (for example one or more paddles) for promoting homogenisation of the vessel contents. Homogenisation of the vessel contents can also be done via a liquid pump inducing circulation flow of the culture medium inside the vessel.

The biological reactor according to the invention is suitable for the culture of photosynthetic microorganisms, in particular for a culture comprising at least one culture step under autotrophic culture conditions comprising water, an inorganic carbon source as well as a light source.

The invention is particularly suitable for culture comprising at least one step under autotrophic culture conditions comprising water, an inorganic carbon source and a light source.

In the scope of the invention, the biological reactor comprises illumination means. The light supply can be made continuously or cyclically (WO2019/034792, WO2021/160776).

The stirring speed and the amount of light as well as the lighting mode can be adapted by the operator in order to obtain optimal culture conditions depending on the geometry of the reactor and the microorganism(s) cultured.

It should be noted that the reactor according to the invention may be equipped with other devices or apparatuses used for controlling and regulating culture conditions of the microorganisms. Mention may be made in particular of the use of probes or sensors for measuring different culture parameters such as temperature, pH, pressure at the inlet and outlet of the reactor.

### Enzymatic Conversion Module

By enzymatic conversion, it is meant transformation of carbon dioxide (CO2) into bicarbonate ions (HCO3-) by an enzyme capable of performing such a transformation, known as conversion enzymes.

The conversion module is a device coupled to the biological reactor and comprising CO2 to HCO3- conversion enzymes immobilised on a support.

Advantageously, controlling the proportion of CO2 dissolved in the form of HCO3- ions makes it possible to control the pH of the reaction medium.

The conversion module is coupled to the biological reactor by any suitable means for circulating the CO2 enriched culture medium in the conversion module and then to the reactor, once the culture medium is enriched with HCO3-.

The reactor and the conversion module form a circuit in which the CO2 enriched culture medium circulates through the conversion module where CO2 is converted to HCO3- then from the conversion module to the reactor.

According to one preferred embodiment, the reactor and the conversion module form a circuit in which the CO2 enriched culture medium circulates from the reactor to the conversion module where CO2 is converted to HCO3- then from the conversion module to the reactor once CO2 is converted to HCO3-. This embodiment allows for more precise control of all reactions, namely dissolution of CO2 in the reactor and conversion of CO2 to HCO3- in the conversion module, so that each parameter such as temperature or flow rate can be optimised. In addition, in the case CO2 dissolving is separated from the culture medium, it is necessary to provide an additional vessel must be provided to dissolve CO2. The present embodiment, in which CO2 is directly dissolved in the culture medium, makes it possible to circumvent this drawback.

According to a still preferred embodiment, the reactor, the conversion module and the CO2 supply device form a circuit in which the culture medium circulates from the reactor to the CO2 supply device, then once enriched with CO2 circulates from the reactor to the conversion module where CO2 is converted to HCO3- and finally once enriched with HCO3- circulates from the conversion module to the reactor once CO2 is converted to HCO3-.

The enzymatic conversion module (53) corresponds to a closed enclosure comprising at least one inlet and one outlet for the circulation of the CO2 receiving fluid, or in one embodiment of the culture medium, the conversion enzymes are immobilised on a support. It is understood that the enclosure may comprise one or more supports comprising said immobilised enzymes, said supports then being arranged randomly or according to a particular organisation in the enclosure.

Dimensions of the enzymatic conversion module, and in particular the amount of enzyme sufficient to obtain a culture medium enriched with HCO3- ions, depending on the method parameters including the enzymatic activity of enzymes used, the CO2 content of the CO2 enriched culture medium and the circulation rate of the culture medium can be adjusted by the operator.

### Enzymatic Conversion Enzymes

Conversion enzymes are known, in particular carbonic anhydrases (CA), and more particularly cadmium-carbonic anhydrases (CDCA) and receptor-type tyrosine phosphatases (TPTR) β and γ.

Carbonic anhydrases are enzymes known for their ability to catalyse the hydration reaction of carbon dioxide (CO2) into bicarbonate ions (HCO3-) in a reversible manner. There are 7 classes: α-AC, β-AC, γ-AC, δ-AC, ζ-AC, η-AC, θ-AC and ι-AC.

Carbonic anhydrases used in the invention are preferably carbonic anhydrases of the α-carbonic anhydrase family.

Such conversion enzymes are especially described in application WO 2013/022348.

In the scope of the present application, enzymes employed according to the invention are selected from cytoplasmic and membrane carbonic anhydrases. Some carbonic anhydrases are commercially available such as bovine erythrocyte carbonic anhydrases marketed by Sigma-Aldrich® (CAS 9001-03-0) or mouse carbonic anhydrases IX marketed by Acrobiosystems®.

Like most enzymes, carbonic anhydrases have a limited lifetime. In order to increase their lifetime, these enzymes may, according to one preferred embodiment of the invention, be used in combination with a proton acceptor. A proton acceptor allows regeneration of a carbonic anhydrase via the formation of a complex so that the carbonic anhydrase can release a proton from its active site and perform a new cycle of conversion from CO2 to HCO3-.

Said proton acceptor is preferably selected from asparagine, glutamine, histidine, serine, threonine, tyrosine, aspartic acid and glutamic acid, even more preferably said proton acceptor is aspartic acid or glutamic acid. The amount of proton acceptor to be used can be adapted by the operator, in particular so that the proportions of proton acceptors and enzymes are stoichiometric.

In such circumstances, all enzymes are regenerated without further interactions between proton acceptors and third-party elements being possible.

### Immobilisation of Conversion Enzymes

Enzyme immobilisation techniques may be based on adsorption, covalent bonding, entrapment or membrane containment.

Several types of supports are suitable for immobilising enzymes. Mention will be made in particular of polymers, especially in the form of a hydrogel or fibres, such as carbon fibre reinforced polymer (CFRP), polyvinylidene fluoride (PVDF), polypropylene (PP), polyacrylonitrile (PAN), silicone and its derivatives, cellulose and its derivatives, such as cellulose acetate, as well as inorganic compounds such as aluminium oxide (Al₂O₃) or even glass or carbon nanotubes.

In one particular embodiment, the enzymes are immobilised in the form of an enzyme precipitate on a support of the fibre type, and more precisely on nanofibres.

The nanofibres may be of natural origin such as collagen-, cellulose-, keratin- or gelatin-based nanofibres, or of artificial origin such as nanofibres based on poly(lactic-co-glycolic acid) (PLGA), poly(3-hydroxybutyrate-co-3-hydroxyvalerate) (PHBV) or poly(ethylene co-vinylacetate).

In another embodiment, the support on which the enzymes are immobilised is an inorganic support, preferably glass. The support can have various geometric shapes: square, rectangle, circle, triangle, polygon or helical helix. The support is possibly a rectangular plate or glass tube. It is meant herein by glass plate or tube, a plate or tube of a macroscopic size. For example, a glass plate with an area of at least 1 cm2, preferably at least 2 cm2, with a length of at least 1 cm, preferably at least 1.25 cm, a width of at least 0.5 cm, preferably at least 0.8 cm and a height less than 0.5 mm, preferably less than 0.13 mm may be used.

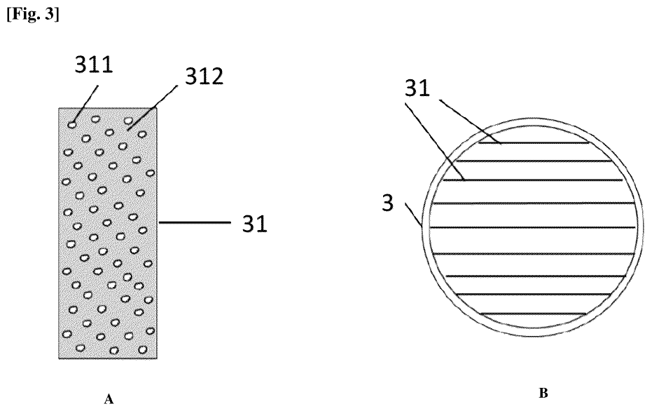

In the case of a glass support, such as a glass plate, and as represented in FIG. 3A, the enzymes 311 can be immobilised using a coating 312 provided on the plate 31. For example, enzymes can thus be immobilised by disulfide bonds, in particular via cysteine molecules using a silanisation process. Trimethoxysilane is then deposited onto the glass surface and cysteine-capped silver nanoparticles are added to form a cysteine layer capable of binding to enzymes. The enzymatic conversion module may then comprise one or more plates, in particular of glass, on which enzymes are immobilised. The plates 31 are preferably arranged in parallel to one another and placed inside the structure 3, as represented in FIG. 3B. It is noted that, in accordance with the representation of FIG. 3B, the structure 3 may have a tubular structure.

When the support comprises a plurality of glass plates, the distance between the latter is at least 2 mm, preferably 4 mm. In a preferred configuration, the plates are preferably spaced from 2 mm to 10 mm, preferably from 4 mm to 6 mm. Enzymes are then advantageously immobilised on each surface of these glass plates, and the culture medium can then circulate between the plates, thus increasing CO2/enzyme contacting. Thus, conversion from CO2 to HCO3- is improved.

In addition, the use of a glass plate can limit the deposit of biomass on the substrate. Consequently, this limits fouling of the culture system. In addition, the use of a glass plate allows for an optimal surface coverage rate and therefore easier access to enzymes. Indeed, it is known to use hollow nanofibres as a support for enzymes, but this type of system experiences rapid fouling, and the enzyme surface coating rate is not satisfactory. This results in a disadvantageous reduction in system efficiency.

The enclosure may comprise one or more supports comprising said immobilised enzymes. With reference to FIG. 3B, the glass plates can especially be arranged in parallel to one another within the conversion module.

### CO2 Supply Device

The CO2 supply device allows delivery of CO2 into the culture medium.

The CO2 supply device may be disposed in the reactor, or outside the reactor on the circulation circuit of the culture medium, upstream or downstream of the conversion module in the direction of circulation.

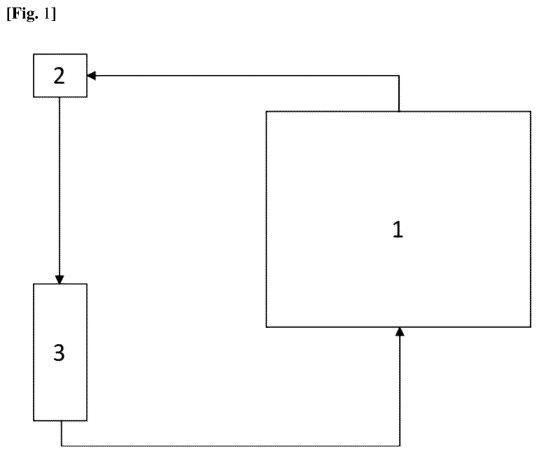

According to a preferred mode, the CO2 supply device is arranged between the reactor and the enzymatic conversion module, upstream of the conversion module in the direction of circulation of the culture medium (FIG. 1).

Many CO2 supply devices exist, including membranes, membrane bubblers or bubble columns, ceramic bubblers, or even bubble diffuser stones.

According to one particular embodiment, the CO2 supply device is a membrane contactor.

According to one preferred embodiment of the invention, when the CO2 supply device is a membrane contactor, it is arranged, in the direction of circulation of the culture medium, after the reactor and before the conversion module.

Parameters for implementing the CO2 supply device adapted to obtain a culture medium enriched in CO2 are adapted to the method parameters including CO2 content of the culture medium before enrichment, kinetics of conversion of CO2 into HCO3- and circulation rate of the culture medium.

### Membrane Contactor

A membrane contactor is a device comprising at least two fluid circulation circuits separated by a transfer membrane. Fluids circulating in said membrane contactor are also referred to as circulation fluid(s). Thus, the membrane contactor comprises a first circulation circuit of a first circulation fluid for the supply of carbon dioxide (CO2), called CO2 supply fluid in contact with a first face of the transfer membrane, and a second circulation circuit of a second circulation fluid for the reception of CO2, called CO2 receiving fluid, in contact with the second face of the transfer membrane. Each fluid circulation circuit is independent of each other, and can be closed or open.

In one embodiment, the circulation circuit of the CO2 receiving fluid is a closed circuit. In another embodiment, the CO2 receiving fluid circulation circuit and the CO2 supply fluid circulation circuit are closed circuits to ensure optimal conditions for the growth of microorganisms.

In one embodiment, the CO2 supply fluid is in the form of gas, preferably an air/CO2 mixture comprising at least 5% CO2, or at least 25% CO2, more preferably at least 50% CO2 and still more preferably at least 75% CO2. Advantageously, the supply fluid is CO2 gas the content of which is 100% CO2.

In another embodiment, the CO2 supply fluid is in the form of a liquid comprising a CO2 absorbing solvent in which CO2 is dissolved.

CO2 absorbing solvent means solvents whose chemical composition allows absorption of CO2 molecules. These solvents are generally solutions comprising carbonate ions (C032-), bicarbonate ions (HCO3-), primary, secondary or tertiary amines or even amino acids. Such solvents are for example solutions comprising β-alanine, sarcosine, taurine, 6-aminohexanoic acid, monoethanolamine (MEA), diethanolamine (DEA), methyl diethanolamine (MDEA), or carbonate compounds such as potassium carbonate (K2CO3), sodium carbonate (Na2CO3) or ammonium bicarbonate (NH4HCO3). These compounds are preferably present in solutions absorbing CO2 at concentrations between 0.1 and 1.0 M.

The CO2 receiving fluid is the culture medium. Said CO2 receiving fluid may also comprise cultured photosynthetic microorganisms and nutrients necessary for their growth.

Thus, according to the invention, the membrane contactor makes it possible to exchange CO2 molecules between a CO2 supply fluid, preferably in the form of a gas, and the CO2 receiving fluid, which is the culture medium, through a membrane, also called a transfer membrane.

Within the membrane contactor, the two fluids, namely CO2 supply fluid and CO2 receiving fluid, are circulating.

Circulation of the CO2 supply fluid can be ensured by different means, including using a CO2 source already under pressure or by means of a gas compressor that ensures a smooth flow through the membrane contactor. In parallel, the circulation of the CO2 receiving fluid, i.e. the culture medium, is permitted thanks to a stirring or agitating element in the biological reactor (for example thanks to the rotation of paddles in the biological reactor) or via pumping of the culture medium which is then reinjected into the biological reactor using a pump. A vacuum pump is then preferably used.

Advantageously, the use of a membrane contactor according to the invention allows a process of injecting CO2 that does not generate harmful shear stresses in the reactor which avoids formation of foam in the culture.

In one embodiment, the flows of the CO2 supply fluid and the CO2 receiving fluid are parallel. In another embodiment, the flows are perpendicular. Still in another embodiment, the flows are cross-flows. The flows of the CO2 supply fluid and the CO2 receiving fluid may be cross-flows, i.e. the CO2 supply fluid and the CO2 receiving fluid circulate in opposite directions.

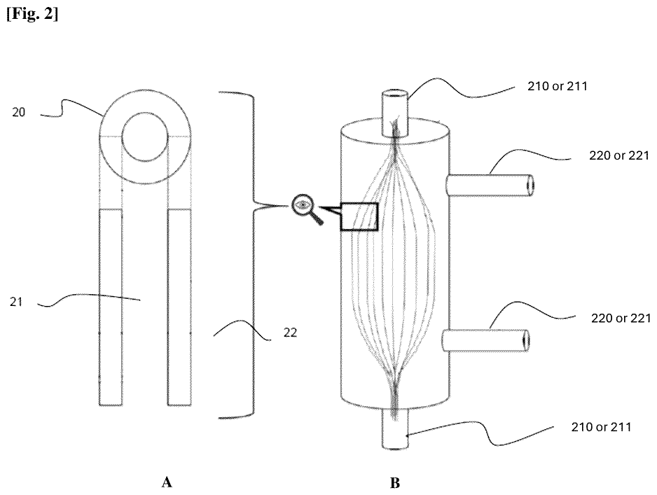

With reference to FIG. 2, the membrane contactor thus comprises an inlet (210) or (211) and an outlet (210) or (211) for a first circulation fluid, as well as an inlet (220) or (221) and an outlet (220) or (221) for a second circulation fluid. These two fluids are separated by a transfer membrane (20). The membrane contactor therefore also comprises two circulation circuits: a first circulation circuit of a first circulation fluid in contact with a first face of the membrane and a second circulation circuit of the second circulation fluid in contact with a second face of the membrane.

The membrane thus allows promoting contact between the two circulation fluids without having direct physical contact. Properties of the membrane determine the nature and characteristics of exchanges between the two circulation fluids.

Membrane contactors are thus defined as systems comprising one or more transfer membranes and whose objective is to promote contacting between two circulation fluids, without having direct contact between the two circulation fluids.

The membrane may be a porous or non-porous membrane also called dense or pervaporation membrane. Dense membranes are membranes without porosity created. They are divided into two categories: hydrophilic membranes and organophilic membranes.

Hydrophilic membranes allow water to pass therethrough and thus, for example, a solvent to be dehydrated. Typically, these membranes are of polymeric material (polyvinyl alcohol) deposited on a porous substrate. Their geometric characteristics are identical to those of ultrafiltration membranes, for example. These membranes are implemented in mainly flat modules, the “permeate” compartment being communicated with a partial vacuum chamber, so as to maintain the acting force and discharge the separation products.

The organophilic membranes are essentially based on silicones (PDMS polydimethylsiloxane) and, in this case, it is the affinity of the silicone material for one or more of the compounds of the mixture that allows selective transfer through the membrane. This results in very high selectivities, with transfer flows generally remaining low.

Membrane contactors are characterised by the transfer membranes that make them up, more particularly by the geometric configuration and structure of these membranes.

The membranes may be of a planar, spiral, tubular or hollow fibre geometric configuration (i.e. pipes or tubes).

According to one preferred embodiment, the membrane(s) have a geometric configuration of hollow fibres. Advantageously, the membrane contactor comprises several fibres dispersed as tube bundles. The external diameter of the membranes is greater than 50 μm, preferably greater than 60 μm, even more preferably greater than 70 μm. In another embodiment, the membranes with a hollow fibre configuration have an external diameter of at least 100 μm. In another embodiment, the external diameter of the membranes is less than 2 mm, preferably less than 1.5 mm, more preferably less than 1 mm. Advantageously, the external diameter of the membranes with a hollow fibre configuration is between 100 μm and 1 mm. The internal diameter of the membranes according to the invention is preferably less than 900 μm, or even less than 850 μm. In one preferred embodiment, the external diameter of the membranes with a hollow fibre configuration is between 100 and 800 μm.

The internal diameter of the membranes with a hollow fibre configuration is generally less than 1 mm, preferably less than 900 μm, less than 800 μm, less than 700 μm, less than 600 μm, less than 500 μm, less than 400 μm, less than 300 μm, less than 200 μm, or even less than 100 μm. In one embodiment, the internal diameter of the membranes with a hollow fibre configuration according to the invention is between 1 and 100 μm. In another embodiment, the internal diameter is between 5 and 100 μm, preferably between 7 and 100 μm, even more preferably between 10 and 100 μm. Advantageously, the internal diameter of the membranes with a hollow fibre configuration according to the invention is between 20 and 90 μm.

With reference to FIG. 2B and according to one embodiment, the membrane contactor comprises a transfer membrane in hollow fibre configuration, the latter being dispersed in the form of a tube bundle. With reference to FIG. 2A, the membrane (20) is in a hollow fibre (or tube) configuration with an internal diameter and an external diameter. Thus, an inner face and an outer face of the membrane corresponding respectively to the internal face of the fibre (or tube) (21) and to the external face of the fibre (or tube) (22) can be defined.

In one embodiment, the CO2 supply fluid circulates within the fibres constituting the membrane and is therefore in contact with the inner face of the membrane, while the CO2 receiving circulation fluid is in contact with the outer face of the membrane. In a second preferred embodiment, the CO2 supply circulation fluid is in contact with the outer face of the membrane and the CO2 receiving circulation fluid is inside the fibres and is in contact with the inner face of the membrane.

In the scope of the present application, the transfer membranes with a hollow fibre configuration are microporous membranes or non-porous membranes.

In one embodiment, the transfer membrane of the membrane contactor is a porous membrane, and comprises pores with a diameter of less than 10 μm. Advantageously, the porous membrane is microporous, and has pores with a diameter of less than 8 μm, preferably less than 5 μm, or even less than 3 μm, more preferably less than 2 μm. In another embodiment, the microporous membrane comprises pores whose diameter is greater than 0.001 μm, 0.002 μm, 0.003 μm, 0.004 μm, or even greater than 0.005 μm. Advantageously, the diameter of the pores of the membrane is thus between 0,005 and 2 μm. In one embodiment, the diameter of the pores is less than 1 μm, or even less than 0.5 μm, and preferably less than 0.2 μm or even less than 0.1 m. In another embodiment, the diameter of the pores of the membrane is between 0.005 and 0.1 m. Still in another embodiment, the diameter of the pores is between 0.008 and 0.09 μm, preferably between 0.009 and 0.07 μm, or even preferably between 0.01 and 0.06 μm, and more preferably between 0.01 and 0.05 μm. Advantageously, the diameter of the pores of the membrane is about 0.01 μm, 0.02 μm, 0.03 μm, 0.04 μm or 0.05 μm.

In one preferred embodiment, the transfer membrane of the membrane contactor is a dense membrane. The diffusion of molecules is then characterised by a diffusion phenomenon through the membrane via the adsorption of molecules having a high affinity with the membrane. This embodiment has the advantage of decreasing or even preventing clogging of the membrane of the membrane contactor with the different elements transported in the liquid medium (such as for example cellular debris) compared to a porous membrane which has potential adhesion zones on its structure and thus makes it possible to maintain the same performance of gas exchanges through the membrane over the entire duration of the culture method.

According to one embodiment, the transfer membrane is a non-porous membrane with an internal diameter of 150 to 450 μm and an external diameter of 250 to 550 μm.

According to one embodiment, the transfer membrane is a porous membrane with an internal diameter of 150 to 250 μm and an external diameter of 250 to 350 μm and comprising pores with a diameter of 20 to 40 μm.

According to another embodiment, the transfer membrane is a dense membrane with an internal diameter of 350 to 450 μm and an external diameter of 5 to 100 μmm.

Advantageously, the transfer membrane, being porous or dense, in particular dense, has a thickness of less than 1000 μm, preferably from 100 μm to 1000 μm.

The use of a membrane contactor according to the invention makes it possible in particular to ensure all or part of the exchanges between two circulation fluids via the presence of a transfer membrane as defined above. The membrane contactor can therefore also be characterised by the exchange surface area between the two circulation fluids.

According to one embodiment of the invention, the membrane contactor has an exchange surface area between the two circulation fluids of at least 0,003 m2/L of CO2 receiving circulation fluid, preferably at least 0,006 m2/L. In parallel, the exchange surface area between the two circulation fluids is less than or equal to 0,020 m2/L per litre of CO2 receiving circulation fluid, preferably less than or equal to 0,017, or even less than or equal to 0,015 and preferably less than or equal to 0,012 m2/L of CO2 receiving circulation fluid. In another embodiment, the membrane contactor has an exchange surface area between 0,003 and 0,012 m2/L of CO2 receiving circulation fluid, preferably between 0,006 and 0,012 m2/L of CO2 receiving circulation fluid.

Materials used for the production of transfer membranes are generally polymers, such as polyethylene (PE), polypropylene (PP), polysulfone (PSu), polyether sulfone (PES), polyvinylidene fluoride (PVDF), polytetrafluoroethylene (PTFE), polyimide (PI), polyamide (PA), polycarbonate (PC), polyacrylonitrile (PAN), and cellulose acetate (CA). The transfer membranes may also be manufactured from silicone or silicone derivatives. In the scope of the present application, the term silicone comprises inorganic compounds formed of a chain of silicon and oxygen atoms and whose silicon atoms can carry groups. Mention will be made in particular of polymers belonging to the siloxane family, and more particularly polydimethylsiloxane (PDMS) or compositions comprising polydimethylsiloxane (PDMS), in particular a ternary system comprised of PDMS, water and tetrahydrofuran (THF). Advantageously, the transfer membranes of the present application are manufactured from silicone, preferably from PDMS, in particular said membranes are thin membranes of PDMS with a thickness between 0.01 and 1.00 cm in sheet or roll form. Examples of commercial transfer membranes are thin silicone membranes of SSP-M823 type from SSP® or PDMS from Saint-Gobain. Such membrane contactors and their operating principles are known per se. They are commercially available, in particular under references pervélys and 3M™ liqui-Cel.

The combination of the use of a conversion enzyme and a membrane contactor makes it possible to reduce the CO2 concentration, or even to lead to the absence of CO2 molecules, in the culture medium, thereby enhancing differences in CO2 concentrations and pressure between the culture medium constituting the CO2 receiving fluid and the CO2 supply fluid. In this way, the diffusion of CO2 molecules from the CO2 supply fluid to the culture medium is promoted.

In one alternative embodiment, the conversion enzymes are advantageously immobilised inside the membrane contactor. In this embodiment, the membrane contactor also fulfils the role of the enzymatic conversion module. This saves space and costs.

According to this alternative embodiment, the enzymes may be immobilised on plates provided inside the membrane contactor, as previously described, or else deposited directly on the surfaces of the membrane contactor.

### Dioxygen (O2) Discharge Device

In one preferred embodiment of the invention, the reactor is included in a closed circuit so as to isolate the culture from environmental hazards external to the reaction, such as temperature variations or variations in the gas composition of the ambient atmosphere.

In this case, it is preferable to discharge dioxygen (O2) produced during photosynthesis by means of a dioxygen discharge device to promote growth of photosynthetic microorganisms and avoid possible overpressures in the reactor enclosure. This may be one or more valves associated with the reactor to allow discharge of the gases with overpressure.

In the case where the CO2 supply means is a membrane contactor, and in particular when the CO2 supply fluid is a gas, discharge of dioxygen is made at the membrane of the membrane contactor, via a path inverse to that of CO2.

In the case where the CO2 supply means is a membrane contactor, and in particular when the CO2 supply fluid is a liquid, discharge of dioxygen will be made by a suitable means, independent of said CO2 supply membrane contactor, for example by means of one or more valves or with another membrane contactor suitable for discharging dioxygen from the culture medium. A membrane contactor suitable for 02 discharge comprises the same structure as a CO2 supply membrane contactor as described above but with an 02 discharge circulation fluid, i.e. a fluid in which the 02 concentration at the inlet of the switch is lower than that in the culture medium, instead of the CO2 supply fluid.

### Conversion Enzyme Regeneration Device

In one particular embodiment, the culture system further comprises an enzyme regeneration device (531). This regeneration device, placed as a bypass of the main circuit of the culture medium, consists of a module for delivering a proton acceptor, said proton acceptor being preferably selected from asparagine, glutamine, histidine, serine, threonine, tyrosine, aspartic acid and glutamic acid, even more preferably, said proton acceptor is aspartic acid or glutamic acid. Thanks to valves (55), circulation of the culture medium is interrupted while circulation of the proton accepting solution (531) is activated. The solution then passes through the enzymatic conversion module (53) in order to regenerate conversion enzymes. The proton accepting solution circulation cycle can be repeated several times in the enzymatic conversion module instead of the culture medium. The number of regeneration cycles to be applied is determined according to the different culture parameters and characteristics of the enzymes used. Circulation of the proton accepting solution is enabled by a pump (54) placed, in the direction of circulation of the solution, between the enzymatic conversion module (53) and the proton accepting solution delivery module (531) or between the proton accepting solution delivery module (531) and the enzymatic conversion module (53).

### Cleaning Device

In another particular embodiment, the culture system further comprises a device (56) for cleaning the circulation circuit of the culture medium. This device is placed as a bypass of the main circuit of the culture medium and consists of a set of cleaning operations comprising one or more washes with water, a wash with an alkaline solution and a wash with an acid solution. The alkaline solution is preferably a soda based solution, comprising from 0.5 to 5% soda. The acid solution is an aqueous solution based on nitric acid for removing limescale and soda deposits in the circuit. In one embodiment, cleaning the device consists in first injecting water, then an alkaline solution and rinsing the device with water, before injecting an acid solution and rinsing the device again with water. It is understood that the operations may be repeated several times if necessary individually or sequentially. The cleaning solutions circulate through the different elements of the culture system, such as the biological reactor (51), the CO2 supply device (52) or the enzymatic conversion module (53), especially by means of pumps (54) and valves (55). A suitable arrangement of valves and pumps within the culture system can thus allow cleaning of one or more elements in an independent or combined manner. The system elements are optimally arranged for the intended purpose.

### Culture System

The invention also relates to a photosynthetic microorganism culture system comprising a culture system comprising a biological reactor, a carbon dioxide (CO2) supply device and a module for enzymatically converting CO2 to HCO3- ions coupled to the reactor, said conversion module comprising enzymes for converting CO2 to HCO3- immobilised on a support.

In one embodiment, the culture medium is first supplied with CO2 at the CO2 supply device before passing through the enzymatic conversion module where catalysis of CO2 into HCO3- ions occurs.

Especially with reference to FIG. 1, the culture system according to the invention comprises a biological reactor (1), a CO2 supply device (2), said CO2 supply device preferably being a membrane contactor, and an enzymatic conversion module (3) in which enzymes are immobilised on a support.

The CO2 supply device is advantageously a membrane contactor. With reference to FIG. 2 and according to one embodiment, the membrane contactor comprises a transfer membrane (20) in hollow fibre configuration, the latter being dispersed in the form of a tube bundle in the membrane contactor. According to one embodiment, the CO2 supply fluid circulates in a first space (21) corresponding to inside the hollow fibres, the fluid circulating along a flow between the inlet (210) or (211) and the outlet (210) or (211) of the hollow fibre bundle. According to this embodiment, the CO2 receiving fluid circulates in a second space (22) delimited by the watertight walls of the membrane contactor, the fluid circulating along a flow between the inlet (220) or (221) and the outlet (220) or (221) of the membrane contactor.

In another embodiment, the CO2 supply fluid circulates in the membrane contactor, i.e. in a space (22) delimited by the watertight walls of the membrane contactor, the CO2 supply fluid then circulating between the inlet (220) or (221) and the outlet (220) or (221) of the membrane contactor. In parallel, according to this embodiment, the CO2 receiving fluid circulates inside the hollow fibres (21) from the inlet (210) or (211) to the outlet (210) or (211).

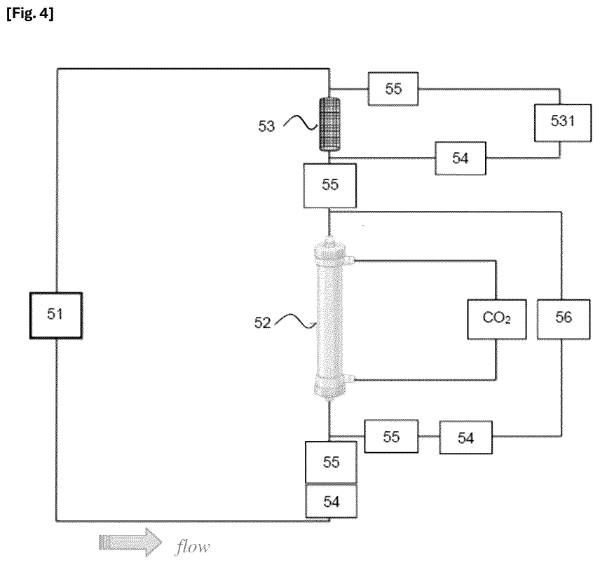

With reference to the embodiment of the invention illustrated in FIG. 4, the microorganism culture system according to the invention comprises a biological reactor (51), a membrane contactor as a CO2 supply device (52) and a module for enzymatically converting CO2 to HCO3- ions (53). The system set forth is preferably a closed circuit.

Preferably, the different elements of the device are arranged as represented in FIG. 4, so that the culture medium first passes through the CO2 supply device (52) and then the enzymatic conversion module (53). According to one embodiment, the culture medium thus circulates from the biological reactor to the membrane contactor (52) where CO2 is delivered, and then the culture medium enriched with CO2 passes through the enzymatic conversion module (53) where catalysis of the CO2 molecules into HCO3- ions occurs, before the culture medium returns to the biological reactor (51).

Preferably, the system may further comprise one or more pumps (54) in order to ensure continuous circulation of the fluids as well as valves (55). It is understood that probes, sensors and other elements for measuring culture parameters or safety systems may be added to the system.

It should be noted that the above embodiments characterised by the presence of a cleaning device and an enzyme regeneration device can be combined with one another and be associated with different CO2 supply devices especially but not only a membrane contactor.

In particular, FIG. 4 shows a specific embodiment comprising as a CO2 supply device a membrane contactor, a cleaning device and an enzyme regeneration device.

It is understood that the culture system according to the invention comprising a biological reactor (1), a CO2 supply device (2) and an enzymatic conversion module (3) is employed for the culture of photosynthetic microorganisms comprising at least one step under autotrophic culture conditions, preferably only steps under autotrophic culture conditions.

### Examples

5 distinct cultures of Chlorella sorokiniana are carried out in a 2.5 L photobioreactor, under artificial lighting with a wavelength between 400 and 460 nm and between 620 and 700 nm and an intensity of 350 μmol/m2/s.

These cultures differ from each other by the use of different CO2 supply devices (bubble or membrane contactor coupled to the reactor) and by the use or not of an alpha-type carbonic anhydrase conversion enzyme from Sigma, CAS: 9001-03-0 with a dosage of 0.1 g/L). The carbonic anhydrase is free in the culture medium or immobilised on a support in an enzymatic conversion module coupled to the biological reactor. The different conditions are summarised in Table 1 below.

TABLE 1

| Culture | CO2 supply device | Carbonic Anhydrase |
|---|---|---|
| A | Bubbler | No |
| B | Membrane | No |
| | contactor | |
| C | Bubbler | Yes, free in the culture medium |
| D | Bubbler | Yes, delivered through the |
| | | use of conversion module |
| E | Membrane | Yes, delivered through the |
| | contactor | use of conversion module |

When a device coupled to the reactor is used (conversion module or membrane contactor, i.e. experiments B, D and E) and there is therefore a circuit in which the culture medium circulates, the flow of the culture medium is induced by a paddle agitator with a speed of 500 revolutions per minute (rpm). It should be noted that homogenisation of the medium is ensured using the same agitating paddle at 500 rpm.

Optical density (OD) measurements are made in duplicate at 750 nm with a spectrophotometer (7315, JENWAY).

In addition, the daily productivity is calculated from the OD and a correlation coefficient (CC) specific to the microorganism cultured according to the following formula, in particular when the biomass concentration is too low to have an accurate value of the solids content.

$$Productivity ( OD ) = ( g . {L}^{- 1} . {day}^{- 1} ) = ( ( OD ( day D ) - OD ( day D - 1 ) ) \times 0.6 ) / ( time ( day D ) - time ( day D - 1 ) ) \times 24$$

That is for the present Chlorella sorokinina culture:

$$Productivity ( OD ) = ( g . {L}^{- 1} . {day}^{- 1} ) = ( ( OD ( day D ) - OD ( day D - 1 ) ) \times 0.6 ) / ( time ( day D ) - time ( day D - 1 ) ) \times 24.$$

1-9. (canceled)

10. A method for culturing photosynthetic microorganisms with HCO₃— ions as the main carbon source in a culture system comprising a biological reactor, a carbon dioxide (CO₂) supply device and a module for enzymatically converting CO2 to HCO₃— ions coupled to the reactor, said method comprising the steps of:
enriching the culture medium with CO₂ via the CO₂ supply device, to obtain a CO2 enriched culture medium, and
contacting the CO₂ enriched culture medium obtained with CO₂ to HCO₃— conversion enzymes in the conversion module to obtain a HCO₃— ion enriched culture medium, wherein the CO₂ to HCO₃— conversion enzymes in the conversion module are immobilised on a support, and wherein the support is a glass plate.

11. The method according to claim 10, wherein the conversion enzymes are carbonic anhydrases.

12. The method according to claim 10, wherein the CO₂ supply device is a membrane contactor.

13. The method according to claim 10, wherein the culture medium meets the autotrophic culture conditions.

14. A photosynthetic microorganism culture system comprising a biological reactor, a carbon dioxide (CO₂) supply device and a module for enzymatically converting CO₂ to HCO₃— ions coupled to the reactor, said conversion module comprising enzymes for converting CO₂ to HCO₃— immobilised on a support, wherein the support is a glass plate.

15. The system according to claim 14, wherein the enzymes are carbonic anhydrases.

16. The system according to claim 14, wherein the flow of the culture medium is oriented from the biological reactor to the CO₂ supply device, then from the CO₂ supply device to the enzymatic conversion module.

17. The system according to claim 14, wherein the carbon dioxide (CO₂) supply device is a membrane contactor.

18. The system according to claim 14, wherein the device further comprises an enzyme regeneration device and/or a cleaning device.
